(19) United States
(12) Patent Application Publication
(10) Pub. No.: US 2022/0022426 A1
(43) Pub. Date: Jan. 27, 2022

(54) DEVICE FOR MONITORING THE STATUS OF A LIVESTOCK FACILITY

(71) Applicant: SoundTalks NV, Leuven (BE)

(72) Inventors: Dries BERCKMANS, Leuven (BE); Zhao Ying CUI, Veltem-Beisem (BE)

(73) Assignee: SoundTalks NV, Leuven (BE)

(21) Appl. No.: 17/295,823

(22) PCT Filed: Dec. 18, 2019
(86) PCT No.: PCT/EP2019/085896

§ 371 Date: May 20, 2021

(51) Int. Cl. A01K 29/00; A01K 15/00; G08B 5/36; H04R 3/00

(52) U.S. Cl. CPC A01K 29/005; A01K 15/00; H04W 84/18; H04R 3/005; G08B 5/36

(57) ABSTRACT

A device for monitoring the status of a livestock facility is disclosed, wherein the status of the livestock facility includes health and/or welfare of livestock animals inside the livestock facility, and management status of various devices installed in the facility. The device includes a housing unit, one or more temperature sensors configured to measure the air temperature in the livestock facility, one or more relative humidity sensors configured to monitor the relative humidity of the air in the livestock facility, one or more light sensors configured to measure the color and/or the light intensity inside the livestock facility to distinguish the real or artificially imposed day and night regimes in the livestock facility, one or more light emitting means configured to indicate the status of the device and/or abnormalities related to livestock animals inside the livestock facility, one or more microphones, one or more loudspeakers, a communication module comprising one or more wireless communication means to interact with other devices.

### FIELD OF INVENTION

The present invention is related to monitoring the status of a livestock facility. More specifically, the present invention relates to utilizing one or more microphones in combination with a plurality of sensors for identifying the abnormalities in a livestock facility.

### BACKGROUND

Diseases in livestock are very common like in humans but identifying diseases in livestock is very difficult. One of the methods is to perform inspection on daily basis which is a very complex and inefficient process as there are large numbers of livestock animals in the farm and to inspect each and every livestock animal requires enormous amount of time and labor which eventually increases the cost.

Use of sensors in monitoring the commercial livestock facilities is more and more common these days. Sensors play an important role in monitoring and tracking of livestock facilities. The administrator/farmer can easily identify whether there is any problem in the farm by analyzing sensor data and comparing it with the predetermined data stored in the database. However the accuracy and precision is still a big concern in the industry.

CN102378981A discloses a system and method for tracking the health of a group of livestock. It employs various sensors like acoustic sensors (microphone), vitality meters, movement sensors, temperature sensors, LED's etc., to collect information related to the group of livestock. Further, collected information is compared to pre-stored data to find existence and/or progress of a disease in the group of livestock. EP2783629A1 discloses a method and/or system for monitoring the cough sounds of cattle with a microphone connected with a computing device, in which the computing device records the sounds made by the animals, performs filtering operations to filter off background noises. Further, it counts the number of sounds caused by respiratory distress over a certain period of time and alerts an operator, if the registered number of sounds caused by respiratory distress exceeds a given value.

The existing system and solutions for monitoring the livestock facilities have several disadvantages:

A disadvantage is that the existing systems do not take into consideration the external environment of the barn/livestock facility affecting health and/or welfare of the livestock animals.

Another disadvantage of the system is interaction, the system comprises of only sending alerts to the user devices in case of abnormalities. The prior techniques do not allow indicating the status of the livestock facilities in general.

Another disadvantage is that the system uses only single microphone to sense the sound of the livestock animals which leads to high probability of error during the analysis or livestock health and/or welfare prediction.

An additional disadvantage is that the analysis is focused only on issues faced by the livestock animals not on the components inside the livestock facilities like heater, ventilation system, feeding lines etc.

There is a need in the art to provide improved techniques of monitoring the commercial livestock facilities for accurately determining health and/or welfare of the livestock animals in the facility along with managing their environment. There is also a need in the art to provide for better techniques of interaction and communication with the monitoring devices for maintaining the health and/or welfare of livestock animals.

### OBJECT OF THE INVENTION

Accordingly, it is a prime objective of the present invention to overcome the above mentioned disadvantages of the prior art by providing a device that utilizes one or more microphones in combination with a plurality of sensors to provide monitoring of a livestock facility.

For use of the device, the device is to be placed inside a livestock facility. Another objective of the present invention is to provide a precise and accurate monitoring of the livestock facility for identifying the abnormalities in the livestock facility.

Another objective of the present invention is to provide improved interaction techniques between the device and the user for maintaining the status of a livestock facility.

Another objective of the present invention is to provide interaction between the device and livestock animals wherein a loudspeaker may be utilized to play calming sounds such as, for example, natural vocalizations of a sow or classical music as a response to aggression detection.

Another objective of the present invention is to provide a device for monitoring the status of a livestock facility using one or more light emitting means on the device to indicate the status of the device and/or abnormalities related to livestock inside the livestock facility.

Another objective of the present invention is to provide a device for monitoring the status of a livestock facility using loudspeaker and one or more microphones such that the positioning of microphone and loudspeaker facilitates automated testing of the microphone.

Another objective of the present invention is to provide monitoring of the livestock facility using plurality of sensors where interdependency of plurality of sensors data is utilized to identify the status of the livestock facility.

Another objective of the present invention is to measure the acoustics of a livestock facility using the combination of microphones and loudspeaker.

### BRIEF DESCRIPTION OF DRAWINGS

In the drawings:

FIG. 1 illustrates a pictorial representation of exemplary device for monitoring the status of a livestock facility.

FIG. 2 illustrates the bottom view of the device.

FIG. 3 illustrates the cross sectional view of the device.

FIG. 4 illustrates an enlarged image of a microphone.

FIG. 5 illustrates various modules present in the device.

FIG. 6. shows a flowchart of the steps followed by the processing module for monitoring the status of a livestock facility.

FIG. 7 illustrates a sound interaction mechanism between the device and the user.

FIG. 8 illustrates a schematic representation of a system for monitoring the status of a livestock facility.

### DETAILED DESCRIPTION

While this solution may be subject to various modifications and take alternative forms, it has been illustrated as an example in the accompanying drawings and will be described in detail below. However, it should be understood that this solution is not intended to be limited to the specific forms disclosed.

Some aspects comparable in terms of scope, the disclosed embodiments are described below. It should be understood that these aspects are presented merely to provide the reader with a brief summary of certain forms that the solution could take and that these aspects are not intended to limit its scope. Indeed, this solution can encompass a variety of aspects that may not be defined below.

As used herein, the terms user, individual user, and individual may be used interchangeably with each other. The present invention is also applicable to “one or more individual users” as described in the claim, includes plurality of users, individual users, individuals.

The term “user” may indicate an owner of the facility and can be any “farmer”, “producer”, “integrator”, “veterinarian” or “administrator” responsible for taking care of the animals on the livestock facility.

The term “livestock animal” may include “cattle”, “pigs”, “horses”, “goats”, “poultry”, “pets” and any animal which can be raised in livestock facilities.

The term “livestock facility” may be used interchangeably with “installations” or “barn” or “facility”.

The term “airspace” may be used interchangeably with “livestock animal section” or “zone” or “space” or “barn” or “facility”.

The term “mobile terminal” may include a plurality of possible devices, such as tablet, personal digital assistant (PDA), and any sort of mobile computing device known in the art.

The term “health”, as used in the present text, refers to the absence of disease, pain and distress.

The term “welfare”, as used in the present text, refers to how a livestock animal is coping with the conditions in which it lives. A livestock animal is in a good state of welfare if, preferably as indicated by scientific evidence, it is healthy, comfortable, well nourished, safe, able to express innate behaviour, and if it is not suffering from unpleasant states such as pain, fear, and distress.

The term “thermal discomfort”, as used in the present text, refers to temperature shock, heat stress and/or cold stress. For example, pigs cannot cope with a temperature shock corresponding to a temperature decrease of 4° C. in one hour.

The term “heat stress”, as used in the present text, refers to a situation where too much heat is absorbed by a person, a plant or an animal, preferably a livestock animal, and causes stress, illness or even death. Heat stress occurs when a body cannot cool itself enough to maintain a healthy temperature. Heat stress is manifested by elevated body temperature, hot, dry skin, lack of sweating and/or neurological symptoms such as paralysis, headache vertigo and/or unconsciousness.

It can also cause heat cramps, heat exhaustion and heat stroke, which may lead to death.

The term “neural networks”, as used in the present text, refers to a network typically comprising an input layer, possibly a number of hidden layers and an output layer each containing different units. The input can be either a set of features or raw audio signals from multiple microphones. An artificial neural network is able to detect patterns in the input data, can extract or identify new useful features, can learn to perform classification tasks, spatial localization of sound events, dereverberation and denoising.

In a first aspect, the present invention relates to a device for monitoring the status of a livestock facility, wherein the status of the livestock facility includes health and/or welfare of livestock animals inside the livestock facility and/or management status of external systems installed in the facility, the device comprising:
i. a housing unit comprising:
   a) one or more temperature sensors configured to measure the air temperature in the livestock facility;
   b) one or more relative humidity sensors configured to monitor the relative humidity of the air in the livestock facility;
   c) one or more light sensors configured to measure the color and/or the light intensity inside the livestock facility to distinguish the real or artificially imposed day and night regimes in the livestock facility;
   d) one or more light emitting means configured to indicate the status of the device and/or abnormalities related to livestock animals inside the livestock facility;
   e) one or more microphones; and
   f) one or more loudspeakers; and
ii. a communication module comprising one or more wireless communication means to interact with other devices and/or external systems.

According to an embodiment, the device is configured to interact with one or more servers, which one or more servers are configured to process output received from the sensors. According to an embodiment, output from microphones and/or sensors of multiple devices are combined to cover a big airspace inside a large livestock facility.

In a preferred embodiment, the invention provides a device for monitoring the status of a livestock facility according to the first aspect of the invention, wherein the device further comprises a memory configured to store output produced by the sensors and microphones enclosed in the housing unit. Alternatively, an external server may be used to receive and store said output. Alternatively, a mobile phone may be used to receive and store said output.

In a preferred embodiment, the invention provides a device for monitoring the status of a livestock facility according to the first aspect of the invention, wherein the device further comprises a processing module for processing output received from the microphones to allow to identify the status of the livestock facility. Alternatively, a mobile phone may be used to receive and store said output and for processing said output.

In a preferred embodiment, the invention provides a device for monitoring the status of a livestock facility according to the first aspect of invention, wherein the device further comprises a sound interaction module configured to allow user interaction with the device, the interaction comprising capturing user's voice through a microphone and providing output related to the status of the livestock facility through the loudspeaker, and/or configured to allow interaction of the device with the livestock animals.

In a preferred embodiment, the invention provides a device for monitoring the status of a livestock facility according to the first aspect of invention, wherein the device comprises:
i. a housing unit comprising:
   a) one or more temperature sensors configured to measure the air temperature in the livestock facility;
   b) one or more relative humidity sensors configured to monitor the relative humidity of the air in the livestock facility;
   c) one or more light sensors configured to measure the color and/or the light intensity inside the livestock facility to distinguish the real or artificially imposed day and night regimes in the livestock facility;
   d) one or more light emitting means configured to indicate the status of the device and/or abnormalities related to livestock animals inside the livestock facility;
   e) one or more microphones; and
   f) one or more loudspeakers;
ii. a communication module comprising one or more wireless communication means to interact with other devices and/or external systems;
iii. a memory configured to store output produced by the sensors and microphones enclosed in the housing unit;
iv. a processing module for processing the output received from the microphones to allow to identify the status of the livestock facility.

Inclusion of the memory and the processing module in the device allows processing of said output without needing external means such as mobile phones and/or servers.

In a preferred embodiment, the invention provides a device for monitoring the status of a livestock facility according to the first aspect of the invention, wherein the device comprises:
i. a housing unit comprising:
   a) one or more temperature sensors configured to measure the air temperature in the livestock facility;
   b) one or more relative humidity sensors configured to monitor the relative humidity of the air in the livestock facility;
   c) one or more light sensors configured to measure the color and/or the light intensity inside the livestock facility to distinguish the real or artificially imposed day and night regimes in the livestock facility;
   d) one or more light emitting means configured to indicate the status of the device and/or abnormalities related to livestock animals inside the livestock facility;
   e) one or more microphones; and
   f) one or more loudspeakers;
ii. a communication module comprising one or more wireless communication means to interact with other devices and/or external systems;
iii. a memory configured to store output produced by the sensors and microphones enclosed in the housing unit;
iv. a processing module for processing the output received from the microphones to allow to identify the status of the livestock facility; and
v. a sound interaction module configured to allow user interaction with the device, the interaction comprising capturing user's voice through a microphone and providing output related to the status of the livestock facility through the loudspeaker, and/or configured to allow interaction of the device with the livestock animals.

In a preferred embodiment, the invention provides a device for monitoring the status of a livestock facility according to the first aspect of the invention, wherein the one or more microphones are arranged in a circular peripheries and are configured to capture sounds produced in surrounding environment, wherein the two or more microphones enhance the accuracy of the device to perform additional functionalities, and wherein the loudspeaker of the housing unit is located at the centre of the circular periphery at equal distances from each of the microphones, such that the positioning of microphones and loudspeaker facilitates automated testing of microphones.

In a preferred embodiment, the invention provides a device for monitoring the status of a livestock facility according to the first aspect of the invention, wherein two or more microphones are arranged in one or more circular peripheries and are configured to capture sounds produced in surrounding environment, wherein the two or more microphones enhance the accuracy of the device to perform additional functionalities, and wherein the loudspeaker of the housing unit is located at the centre of the circular periphery at equal distances from each of the microphones, such that the positioning of microphones and loudspeaker facilitates automated testing of microphones.

With two or more microphones, noise sources can be localized better, these sources can then be modelled better and can be better extracted from an incoming audio signal. By doing such pre-processing step better, i.e. by using more than one microphone, the accuracy of for example cough detection is enhanced. In relation to said automated testing of microphones, the one or more circular peripheries of microphones around the loudspeaker makes that a known audio signal sent out of the loudspeaker is picked up by all the surrounding microphones, and in the ideal case, exactly the same signal is picked up by all the microphones. During an audio signal processing step, these signals can be compared to rank the microphones from high to low quality, and a decision can be made to change from one microphone to the other or to disable an extremely bad microphone.

In a preferred embodiment, the invention provides a device for monitoring the status of a livestock facility according to the first aspect of the invention, wherein the sound interaction module is configured to allow microphone quality measurements by playing a predefined sound of known composition by the loudspeaker and recording the sound by the one or more microphones, resulting in one or more microphone signals, and determining the difference between the original sound played by the loudspeaker and the one or more microphone signals, or the correlation between the original sound and the one or more microphone signals, or, when multiple microphones are present, the correlation between pairs of microphone signals.

In a preferred embodiment, the invention provides a device for monitoring the status of a livestock facility according to the first aspect of the invention, wherein the livestock animals in the livestock facility are selected from the group consisting of cattle, pigs, horses, goats, poultry, pets and any animal which can be raised in livestock facilities.

In a preferred embodiment, the invention provides a device for monitoring the status of a livestock facility according to the first aspect of the invention, wherein the livestock animals are one or more pigs.

In a preferred embodiment, the invention provides a device for monitoring the status of a livestock facility according to the first aspect of the invention, wherein the livestock animals are one or more chickens.

In a preferred embodiment, the invention provides a device for monitoring the status of a livestock facility according to the first aspect of the invention, wherein the livestock animals are one or more turkeys.

In a preferred embodiment, the invention provides a device for monitoring the status of a livestock facility according to the first aspect of the invention, wherein the one or more light emitting means comprise one or more multicolor light emitting means.

In a preferred embodiment, the invention provides a device for monitoring the status of a livestock facility according to the first aspect of the invention, wherein the one or more multicolor light emitting means comprise one or more multicolor LEDs.

In a preferred embodiment, the invention provides a device for monitoring the status of a livestock facility according to the first aspect of the invention, wherein the one or more wireless communication means comprise a wireless communication module, a module configured for exchanging data wirelessly over short distances using short-wavelength ultra-high-frequency radio waves in industrial, scientific and medical radio bands from 2.400 to 2.485 GHz, and/or a low energy wireless technology for data exchange over short distances using short-wavelength ultra-high-frequency radio waves in industrial, scientific and medical radio bands from 2.400 to 2.485 GHz. The industrial, scientific and medical radio bands are radio bands (portions of the radio spectrum) reserved internationally for the use of radio frequency (RF) energy for industrial, scientific and medical purposes other than telecommunications. In a preferred embodiment, the wireless communication module is a Wi-Fi module. In a preferred embodiment, the module configured for exchanging data wirelessly over short distances using short-wavelength ultra-high-frequency radio waves in industrial, scientific and medical radio bands from 2.400 to 2.485 GHz is a Bluetooth module. In a preferred embodiment, the low energy wireless technology for data exchange over short distances using short-wavelength ultra-high-frequency radio waves in industrial, scientific and medical radio bands from 2.400 to 2.485 GHz is Bluetooth low energy. In a preferred embodiment, the invention provides a device for monitoring the status of a livestock facility according to the first aspect of the invention, wherein the one or more wireless communication means comprise a Wi-Fi module, a Bluetooth module and/or Bluetooth low energy.

In a preferred embodiment, the invention provides a device for monitoring the status of a livestock facility according to the first aspect of the invention, wherein the one or more wireless communication means comprise a digital wireless data communication technology using sub-gigahertz radio frequency bands like 169 MHz, 433 MHz, 868 MHz and 915 MHz. Said digital wireless data communication technology enables very-long-range transmissions (more than 10 km in rural areas) with low power consumption. Said technology permits inexpensive, long-range connectivity for Internet of Things (IoT) devices in rural, remote and offshore industries, and is typically used in mining, natural resource management, renewable energy, transcontinental logistics, and supply chain management. In a preferred embodiment, the digital wireless data communication technology using sub-gigahertz radio frequency bands like 169 MHz, 433 MHz, 868 MHz and 915 MHz is LoRa. LoRa is presented in two parts, i) LoRa (Long Range), the physical layer and ii) LoRaWAN (Long Range Wide Area Network), the upper layers.

In a preferred embodiment, the invention provides a device for monitoring the status of a livestock facility according to the first aspect of the invention, wherein one section of the housing unit is translucent in nature to indicate one or more different colors of the light emitting means.

In a preferred embodiment, the invention provides a device for monitoring the status of a livestock facility according to the first aspect of the invention, wherein the additional functionalities provided by the plurality of microphones comprise sound source localization, noise reduction, dereverberation, determining directionality of sound, and/or advanced signal processing techniques selected from the list comprising neural networks and a beamforming operation.

In a preferred embodiment, the invention provides a device for monitoring the status of a livestock facility according to the first aspect of the invention, wherein the one or more light emitting means utilize different colors to indicate the status of the device and/or abnormalities related to the livestock animals, the different colors and meaning selected from the following:
a. a color, for example purple, to indicate that the device is not connected to the internet;
b. another color, for example green, to indicate that the device is online and the status is ok;
c. yet another color, for example red, to indicate a potential disease outbreak in the livestock facility;
d. even yet another color, for example yellow, to indicate an intermediate state indicating the need of increased vigilance.

In a preferred embodiment, the invention provides a device for monitoring the status of a livestock facility according to the first aspect of the invention, wherein the device is configured to monitor the health of the livestock animals by monitoring one or more sounds related to disease comprising sounds of and/or sounds related to coughing, sneezing, snicking and/or screaming, and/or the device is configured to monitor the welfare of the livestock animals by monitoring one or more sounds of or sounds related to aggression by humans or animals, burglary, trespassing, boredom and/or thermal discomfort. An example of boredom is a bored state of pigs when lacking a sufficient amount of entertaining activities like eating or playing.

Preferably, the device is configured to monitor the health and/or the welfare of the livestock animals by further comprising a microprocessor, the microprocessor being configured for running algorithms, which algorithms are designed to use inputs from the sensors of the device in order to determine and thus monitor the health and/or welfare of livestock animals, which sensors also include the microphones of the device, which microphones can also be interpreted as acoustic sensors.

In a preferred embodiment, the invention provides a device for monitoring the status of a livestock facility according to the first aspect of the invention, wherein the device is configured to monitor one or more external systems installed in the facility, which one or more external systems are selected from the group comprising feeding lines, drinking systems, sprinklers, ventilation systems, heating systems, cleaning systems, and artificial lights.

In a preferred embodiment, the invention provides a device for monitoring the status of a livestock facility according to the first aspect of the invention, wherein the module configured for exchanging data wirelessly over short distances using short-wavelength ultra-high-frequency radio waves in industrial, scientific and medical radio bands from 2.400 to 2.485 GHz is configured to track the movement of the staff through the livestock facility and is configured to improve the biosecurity by determining the order in which the livestock animals should be visited.

Preferably, in relation to the configuration of the device to improve the biosecurity by determining the order in which the livestock animals should be visited, staff working in a livestock facility are equipped with a beacon while working in a livestock facility or while moving from one compartment of a livestock facility to another compartment of the livestock facility. Preferably, different devices in a livestock facility are equipped with wireless technology able to track the beacon, by sending and receiving signals from one of the devices to the beacon and vice versa. Accordingly, a path the staff is taking can be visualized. Biosecurity is thus for example improved by instructing the staff which livestock animals to visit first, and in particular first the healthy animals and only later on the sick animals.

In a preferred embodiment, the invention provides a device for monitoring the status of a livestock facility according to the first aspect of the invention, wherein the livestock facility further comprises a gateway configured as the internet access point, enabling the creation of a wireless communication means, and preferably a Wi-Fi, mesh network automatically detecting and connecting all devices within reach of the gateway, either directly or indirectly through other devices, or the creation of a network wherein the devices are physically connected to the gateway by a plurality of Ethernet cables.

According to another preferred embodiment, detection and connection of devices, and/or transmittal of sensor and/or microphone output, is effected by a digital wireless data communication technology using sub-gigahertz radio frequency bands like 169 MHz, 433 MHz, 868 MHz and 915 MHz, preferably LoRa, for which a communication means for digital data communication using sub-gigahertz radio frequency bands like 169 MHz, 433 MHz, 868 MHz and 915 MHz, preferably a LoRa communication means, can be used. When utilizing a digital wireless data communication technology using sub-gigahertz radio frequency bands like 169 MHz, 433 MHz, 868 MHz and 915 MHz, preferably LoRa, no gateway is needed.

In a preferred embodiment, the invention provides a device for monitoring the status of a livestock facility according to the first aspect of the invention, wherein the loudspeaker may be utilized to play any type of sound/music for the livestock animals and/or workers, wherein the sound/music is selected from the group comprising a calming sound for the livestock animals, classical music, natural vocalizations of the mother animal and music to improve the work environment of the workers. For example, to reduce symptoms of boredom of one or more pigs, a variety of sounds can be produced to entertain the pigs with sound-based games.

In a preferred embodiment, the invention provides a device for monitoring the status of a livestock facility according to the first aspect of the invention, wherein the gateway is equipped with data storage means, preferably one or more solid state drives and/or USB sticks, to store raw audio recording.

In a preferred embodiment, the invention provides a device for monitoring the status of a livestock facility according to the first aspect of the invention, wherein the memory comprises magnetic storage units, optical storage units, RAM, ROM, hard drives, and/or flash memory.

In a preferred embodiment, the invention provides a device for monitoring the status of a livestock facility according to the first aspect of the invention, wherein the loudspeaker in combination with one or more microphones are configured to measure the acoustics of the livestock facility to distinguish a big livestock facility from a smaller livestock facility.

In a preferred embodiment, the invention provides a device for monitoring the status of a livestock facility according to the first aspect of the invention, wherein the device further comprises a microprocessor and wherein the loudspeaker in combination with one or more microphones are configured to measure the acoustics of the livestock facility by means of said microprocessor, which microprocessor is configured to run an algorithm which can determine acoustics from sounds, and which microprocessor on the basis of the acoustics is configured to distinguish a big livestock facility from a smaller livestock facility. Blind estimation of acoustics is really important for having better accuracy in classification algorithms, for distinguishing big and small farms and to track the growth of the animals, because the acoustics change with growing animals.

In a preferred embodiment, the invention provides a device for monitoring the status of a livestock facility according to the first aspect of the invention, wherein the sound interaction module further allows two way interactions between the user and the device, where the device can ask questions to the user and store a received answer for future reference, and/or where the user can ask questions to the device and receive an answer from the device.

In a preferred embodiment, the invention provides a device for monitoring the status of a livestock facility according to the first aspect of the invention, wherein the housing unit further includes one or more antennas to communicate with other devices, gateway or external systems. External systems can also be called external installations.

In a preferred embodiment, the invention provides a device for monitoring the status of a livestock facility according to the first aspect of the invention, wherein the one or more antennas are placed in a perpendicular plane compared to the position of the one or more microphones to minimize interference noise on the one or more microphones.

In a preferred embodiment, the invention provides a device for monitoring the status of a livestock facility according to the first aspect of the invention, wherein the housing unit further includes one or more gas sensors suitable to measure gasses or emissions, which one or more gas sensors are selected from the group comprising sensors suitable to measure concentrations of ammonia, CO₂, butyric acid, dust and/or odor.

In a preferred embodiment, the invention provides a device for monitoring the status of a livestock facility according to the first aspect of the invention, wherein the housing unit includes a camera, which camera is for example, but not limited to, suitable to detect movement, activity, occupancy of the livestock facility and/or weight of the livestock animals.

In a preferred embodiment, the invention provides a device for monitoring the status of a livestock facility according to the first aspect of the invention, wherein the housing unit further includes an electronic nose suitable to identify specific particles in the air for detecting diseases in livestock animals.

In a second aspect, the present invention relates to a system for monitoring the state of a livestock facility, wherein the status of the livestock facility includes health and/or welfare of livestock animals when inside the livestock facility and/or management status of external systems when installed in the facility, comprising:
a livestock facility; and
one or more devices according to the first aspect of the present invention suitable to be placed inside the livestock facility.

External installations can be used as a synonym for external systems.

In a preferred embodiment, the invention provides a system for monitoring the state of a livestock facility according to the second aspect of the invention, wherein the system further comprises a mobile apparatus, preferably a mobile phone, configured to receive output from the one or more devices, to store said output and to process said output.

In a preferred embodiment, the invention provides a system for monitoring the state of a livestock facility according to the second aspect of the invention, wherein two or more of said devices are distributed inside the livestock facility. Preferably, the two or more of said devices which are distributed inside the livestock facility are connected with each other wirelessly. By using a distribution of two or more of said devices, a full coverage in terms of monitoring can be obtained for all livestock animals inside a livestock facility.

In a preferred embodiment, the invention provides a system for monitoring the state of a livestock facility according to the second aspect of the invention, wherein the system further comprises one or more external systems placed inside the livestock facility.

In a preferred embodiment, the invention provides a system for monitoring the state of a livestock facility according to the second aspect of the invention, wherein the one or more external systems, which can also be called the one or more external installations, are selected from the group comprising feeding lines, drinking systems, sprinklers, ventilation systems, heating systems, cleaning systems, music systems and artificial lights. According to embodiments, the one or more external systems are connected to and/or controlled by the one or more devices and/or a control unit.

In a preferred embodiment, the invention provides a system for monitoring the state of a livestock facility according to the second aspect of the invention, wherein one device according to the first aspect of the invention is placed inside the livestock facility at level of each area inside the livestock facility with a diameter of between 16 and 24 m, more preferably of between 17 and 23 m, even more preferably of between 18 and 22 m, yet even more preferably of between 19 and 21 m, and most preferably of 20 m, which is intended to accommodate livestock animals. For example, 200 to 250 pigs may be accommodated in an area with a diameter of 20 m, so typically 4 devices should be placed in a livestock facility for accommodating 1000 pigs. For example, 4000-6000 chickens may be accommodated in an area with a diameter of 20 m, so typically 5 devices should be placed in a livestock facility for accommodating 25000 chickens.

The disclosure is further described by the following non-limiting figures which further illustrate the disclosure, and which are not intended to, nor should they be interpreted to, limit the scope of the disclosure.

### FIGURES

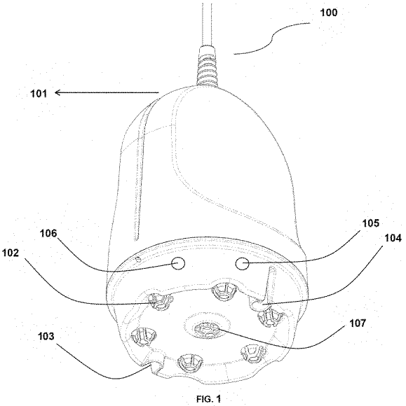

FIG. 1

FIG. 1 illustrates a pictorial representation of exemplary device 100 for monitoring a livestock facility. The device may be positioned at any appropriate position in the livestock facility so as to accurately monitor health and/or welfare of livestock animals and the surrounding environment. The device can be installed easily on the livestock facility by means of powering it through a power cable. The livestock facility comprises heating and cooling (ventilation) systems to regulate the temperature and keep livestock animals in their most thermo-comfortable zone (age related) so they can use all their energy for growth and thus meet production. The livestock facility also comprises feeding line systems for feeding the livestock animals, lights to potentially impose an artificial pattern of day or night to improve the growing of certain species.

As shown in the FIG. 1, the device 100 is made up of a housing unit 101 comprising two or more microphone 102-1 . . . 102-6, herein collectively referred as the microphones 102 and individually referred as microphone 102, a temperature sensor 103, a relative humidity sensor 104, LEDs 105, a light sensor 106, and a loudspeaker 107. In the remainder of the text, “housing unit” and “housing” are used interchangeably. The housing unit 101 is mainly dome-shaped and can be manufactured from polypropylene, polyethylene and/or polyvinylchloride. The housing unit 101 is sturdy and resistant to an environment inside a livestock facility. With resistant is meant that the housing unit 101 can survive harsh circumstances inside a livestock facility, like for example exposure to straw, and exposure to accidental physical impact caused by animals. A smartphone, on the other hand, would not be suitable to do any kind of monitoring inside a livestock facility, since a smartphone cannot survive in a livestock facility, because a smartphone is not resistant to an environment inside a livestock facility because of its less sturdy housing. The housing unit 101 shown in FIG. 1 is connected to an electrical wire for providing electrical energy via connections to said microphones 102, temperature sensor 103, relative humidity sensor 104, LEDs 105, light sensor 106 and loudspeaker 107.

The device comprises two or more microphones 102 for recording the sounds produced in the livestock facility. Preferably the device includes six microphones arranged in a circular periphery on the bottom part of the housing. All the microphones are placed in a plane and are pointing downwards. The microphones are configured to capture the sounds generated by the livestock animals which are later analyzed to determine the health and/or welfare of the livestock animals. The microphones also capture the sounds generated by the various systems including heating systems, ventilation systems, feeding lines, cleaning systems, etc. Microphones 102 provide an indication on health, welfare, and/or management status of livestock facility. One or more sounds related to diseases like, for example, sounds of coughing, sneezing, snicking and screaming, have a sound associated with them and may easily be captured in microphone 102. Issues such as aggression, tail biting, etc can also be associated with certain sounds. Malfunctioning of feeding lines or ventilations or heating systems is audible and thus detectable through microphone 102. Multiple microphones 102 allows for potential extra functionalities such as sound source localization, noise reduction, dereverberation, determining directionality of sound and more advanced signal processing techniques selected from the list comprising neural networks and a beamforming operation.

The temperature sensor 103 is located outside of the housing so it can measure the environmental temperature in the livestock facility. The comfort and growth of livestock animals is very much linked to the temperature they are feeling. If the temperature is too low, the livestock animal will feel cold and they will use energy to generate heat. This means that this energy will not be available anymore for growing. Just like human beings, livestock animals can adapt themselves to changing temperature if the change is gradual. Sudden drops can lead to high intolerance and disease outbreak due to lower biological resistance of the livestock animal. Every age group also has their own comfort temperature. It is therefore important to follow the temperature in a livestock barn for both health and welfare as well as management issues. A sudden drop in temperature due to cold wind from the north blowing on the building can be an alarm for potential disease outbreak, whereas the same drop due to malfunctioning heating or ventilation is clearly a management issue.

The relative humidity sensor 104 positioned opposite from the temperature sensor on the outside of the housing, measures the relative humidity inside the livestock facility. Temperature in combination with the relative humidity determines the feeling temperature. For example, in humans, 30° C. will feel different with 50% humidity or 90% humidity, where the latter will feel less pleasant because it's harder to transfer body heat by sweating. Combination of temperature sensor 103 and relative humidity sensor 104 provides the feeling temperature inside the livestock facility. The combination of temperature and relative humidity provides information on the environment in which livestock animals grow. Deviations from the adequate environment for growing livestock animals can be detected early and possible disease outbreaks can be predicted earlier with this information.

The LEDs 105 are positioned inside the housing and indicate the device status to the user. The colors of the LEDs 105 report both on the status of the hardware device itself as well as data issues. In an example case, six LEDs are positioned inside the housing with a translucent cover for indicating the status of the device in the livestock facility. The color of the LEDs is indicative of the status of the livestock facility such as purple color may indicate that the device is not connected to the internet, green color may indicate that the device is online and the status is ok, red color may indicate that there is a potential disease outbreak in the livestock facility, preferably based on a very high amount of coughing, sneezing, snicking and/or screaming in the livestock facility, yellow color may indicate an intermediate state indicating the need of increased vigilance, etc.

The device comprises a light sensor 106 located in the interior of the bottom translucent part of the housing opposite to the LEDs such that it is not influenced by the light of LEDs. It is configured to measure light intensity inside the livestock facility. The light sensor will be used to keep track of night and day. This can be the natural night and day or an artificial pattern of night and day imposed with lights in order to improve the growing of certain species.

The device 100 comprises a loudspeaker 107 placed in the middle of the bottom part at the center of all the microphones. The placement ensures that it is equidistant from all the microphones and facilitates automatic quality measurements of the microphones. It allows for playing a predefined sound of known composition. When the microphones, which are lying just around it, record this sound and send the recorded sound through resulting in microphone signals, the difference between the original sound played by the loudspeaker and the microphone signals, or the correlation between the original sound and the microphone signals, or the correlation between pairs of microphones, can provide information on the quality of the microphones. The loudspeaker is further configured to play sound for the livestock animals and/or workers, wherein the sound may include but is not limited to a calming sound for the livestock animals, classical music or the sound of natural vocalizations of the mother animal, music to improve the work environment of the workers, etc. The combination of the loudspeaker and microphones makes the device interactive by allowing a user or administrator of the facility to interact using voice based commands. The microphones pick up the voice of the user which is further analyzed by the processor to determine the appropriate response which is played to the user from the loudspeaker. The loudspeaker 107 and the microphones 102 may be further utilized to measure the acoustics of the livestock facility. With two or more microphones in combination with the loudspeaker a model can be made from the acoustics of the livestock facility, and can distinguish a big, reverberant livestock facility from a small, non-reverberant livestock facility. The knowledge about the acoustics of the livestock facility is beneficial for the classification of different sounds. A single device is capable of monitoring a group of livestock animals where in the size of group is determined based on the type of livestock animals, size of barn, environmental conditions . . . etc. In bigger installations multiple devices may be installed in one open space (referred to as air space) which thus enables the owner of the livestock facility to visualize the status of the whole installation on airspace level. Moreover, multiple devices can be placed in a Wi-Fi Mesh network to monitor health and/or welfare in big airspaces.

As an example, a single device may be capable of monitoring 200-250 pigs or 4000-6000 chickens in a barn. In case of barns of more than 250 pigs or 5000 chickens, multiple devices, preferably connected in a Wi-Fi mesh network, may be used for monitoring purpose.

FIG. 2

FIG. 2 illustrates the bottom view of the device which is circular in shape and shows the positioning of the microphones 102, temperature sensor 103, relative humidity sensor 104 and loudspeaker 107. The loudspeaker 107 is located in the center of all the microphones 102. The microphones 102 are placed at equal distance from the loudspeaker which facilitates automated quality measurements. The temperature sensor 103 and relative humidity sensor 104 are placed opposite to each other along a straight line.

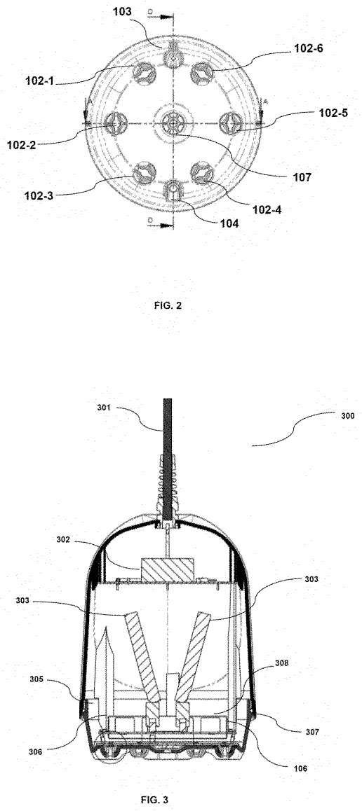

FIG. 3

FIG. 3 illustrates the cross sectional view of the device 100. Device 100 consists of a housing which is divided into two parts i.e. top part and bottom part. The top part is hollow inside and may be connected to bottom part using right locking mechanism 305 and left locking mechanism 307. Top part consists of electric wire 301 and electric power module 302 for supplying the power to the device. The bottom part consists of antenna, LEDs, microphone, temperature sensor, relative humidity sensor, light sensor and loudspeaker. Bottom part further contains a pillar to press down the pcb 306 to hold it in the right place when top and bottom parts get connected. Metal shield 308 is coated around the pcb 306 so that the pcb is not affected by the Electromagnetic wave radiation. The side wall of the bottom part of the housing is made up of translucent material so that status of LEDs can be visible to the user.

The housing unit 101, further includes Wi-Fi Antennas 303 to communicate with other devices wherein the Wi-Fi antennas 303 are placed in a perpendicular plane compared to the position of microphones to eliminate interference noise on the microphones

The light sensor 106 is located in the interior of the bottom translucent part of the housing. It is placed on the opposite side of the LEDs 105 to not be influenced by the light of it.

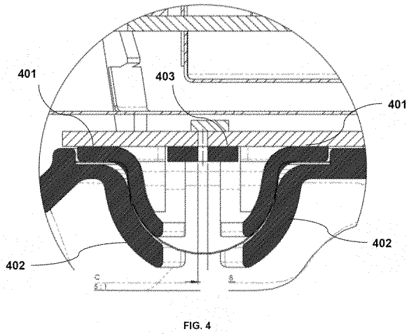

FIG. 4

FIG. 4 illustrates an enlarged image of microphone 102. Each microphone 102 is covered by a protective membrane 401 and structural dome 402. Protective membrane 401 protects the microphone. Structural dome 402 protects the protective membrane 401 from mechanical impact as well as dirt. It also enlarges the surface on which dirt can come without blocking the entrance to the microphone. Sealing gasket 403 is included in the microphone to seal the gaps inside the microphone for optimal acoustic performance.

FIG. 5

FIG. 5 illustrates various modules present in the device 100. As shown in the figure the device comprises a sensing module 501 comprising microphones, which microphones can also be interpreted as acoustic sensors, a temperature sensor, a relative humidity sensor and a light sensor, a communication module 502 comprising LEDs, a Wi-Fi module and/or a Bluetooth module, a memory 503 configured to store output produced by the sensors, processing module 504 for processing output received from the microphone in combination with output received from the other sensors to identify the status of the livestock facility and a sound interaction module 505 configured to allow user interaction with the device, the interaction comprising capturing user's voice through the microphone and providing output related to the status of the livestock facility through loudspeaker, and/or configured to allow interaction of the device with the livestock animals, and/or configured to allow microphone quality measurements by playing a predefined sound of known composition by the loudspeaker and recording and sending through the sound by the microphones, resulting in microphone signals, and determining the difference between the original sound played by the loudspeaker and the microphone signals, or the correlation between the original sound and the microphone signals, or the correlation between pairs of microphones. A non-limiting example of a configuration to allow interaction of the device with the livestock animals is the playing of calming sounds through the loudspeaker in response to detected aggression of the livestock animals.

The sensors in the sensing module monitor the environmental conditions in the livestock facility. The LEDs are configured to indicate the status of the device by indicating a different color for different status.

The Wi-Fi module allows the device to communicate with other devices installed in the facility and/or user devices to provide updates regarding the status of the livestock facility. The livestock facility further comprises a gateway used as an internet access point using wired (Ethernet cable) or wireless connection (4G router). All the devices within reach of the gateway, either directly or indirectly through other devices, will be detected and connected automatically via a Wi-Fi mesh network and/or via a plurality of Ethernet cables suitable to physically connect the devices to the gateway. It is advantageous that the plurality of Ethernet cables can ensure connection of the devices to the gateway when for some reasons the Wi-Fi mesh network would fail working. A mesh network entails that a device that is out of reach of the gateway, but is within reach of another device that is in reach of the gateway, can also connect to the gateway, through the other device. The mesh network is a dynamic network which means that if a device cannot reach the gateway through a certain path, it will try to find another set of devices, through which, it can reach the gateway. The gateway may also be equipped with solid state drives to store raw audio recording.

The Bluetooth module allows tracking the movement of the staff through the barn and improves the biosecurity by determining the order in which the livestock animals should be visited (such as first the younger and healthy livestock animals and only later the bigger and sick livestock animals).

A memory 503 is used to store sensor data locally in the device. The memory may include but is not limited to magnetic storage units, optical storage units, RAM, ROM, hard drives and/or flash memory.

The processing module 504 processes output produced by the various sensors in combination with output produced from the microphones to identify the status of the livestock facility.

The sound interaction module 505 allows the users to interact with the device by giving voice commands to the device which are picked up by the microphones. The device responds to the commands by playing the required answer through the loudspeakers. Additionally, the sound interaction module 505 may allow interaction of the device with livestock animals. A non-limiting example of a configuration to allow interaction of the device with livestock animals is the playing of calming sounds through the loudspeaker in response to detected aggression of the livestock animals. Additionally, the sound interaction module 505 may allow microphone quality measurements by playing a predefined sound of known composition by the loudspeaker and recording and sending through the sound by the microphones, resulting in microphone signals, and determining the difference between the original sound played by the loudspeaker and the microphone signals, or the correlation between the original sound and the microphone signals, or the correlation between pairs of microphones

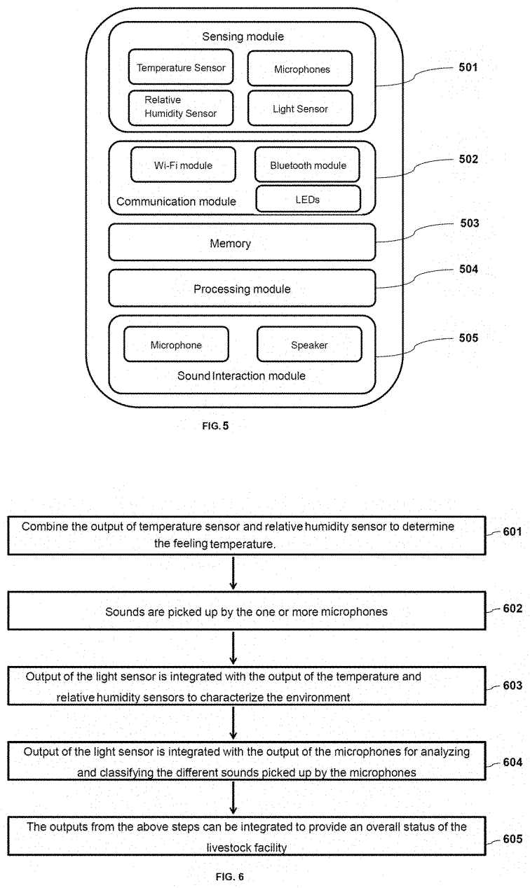

FIG. 6

FIG. 6 shows a flowchart of the steps followed by the processing module for monitoring the status of a livestock facility. A main feature of the device is that it takes into account the interdependency of different sensor values to determine the exact health and/or welfare of the livestock facility. For example, when livestock animals don't calm down within a certain period where it becomes dark then it could indicate something abnormal happening such as a disease or other disturbing factors for the livestock animals. In such scenarios, output of light sensor (indicative of dark in the surroundings) is utilized in combination with output from microphones (indicative of sounds of aggression or disease) and/or temperature or relative humidity sensor (indicative of non-favorable environment or malfunctioning of the heating and/or ventilation systems) to determine the status of the livestock facility and take appropriate measure to mitigate the problems. The processing module thus helps in handling the health and/or welfare as well as management issues on the livestock facility. A sudden drop in temperature due to cold wind can be an alarm for potential disease outbreak, whereas the same drop due to malfunctioning heating or ventilation is clearly a management issue.

As shown in figure, at step 601 output of temperature sensor and relative humidity sensor is combined to determine the feeling temperature. At step 602 various sounds are picked up by the one or more microphones. At step 603 output of the light sensor is integrated with the output of the temperature and relative humidity sensors to characterize the environment in which the livestock animals are growing (cold-warm, humid-dry, day-night). Most livestock facilities have fixed temperature threshold for different times of the days to ensure the healthy growth of livestock animals and keep them in their most thermo comfortable zone. Deviations from this adequate temperature can be detected early and the failures in various temperature regulating systems can be detected more robustly. The findings can be integrated in the health and/or welfare monitoring system for the prediction of possible disease outbreaks based on changing environmental parameters (such as temperature drops) and/or malfunctioning of the various systems installed on the facility for maintaining the temperature (heating and ventilation systems).

At step 604 the output of the light sensor is integrated with output of the microphones for analyzing and classifying the different sounds picked up by the microphones. As an example, the output of the light sensor can be combined with the output from the microphones in order to look for specific events in the night by only listening to the sound during the night. The operations in a livestock facility and the behavior of livestock animals are different during the day as compared to the night. The livestock animals are likely to be more active during the day, compared to the night. Similarly, more feeding lines are operational during the daytime. Deviations from this pattern (i.e. non-active during the day) can also be related to the health and/or welfare of the livestock animals and malfunctioning of the feeding line systems, and can be predicted by combining light sensor and microphones. At step 605, the outputs from the above steps can be integrated to provide an overall status of the livestock facility. The status can be indicated to the user and/or veterinarian using one or more color of the LEDs or communicated as voice response through the loudspeaker.

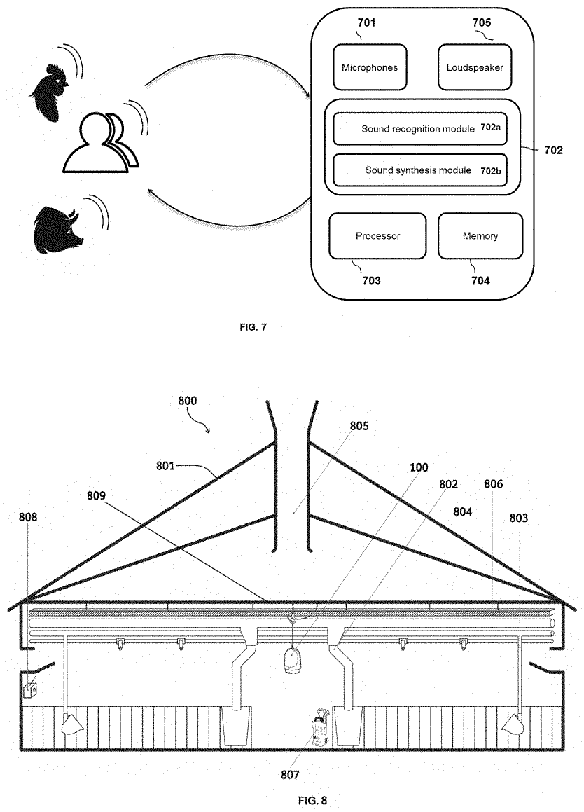

FIG. 7

FIG. 7 illustrates a sound interaction mechanism between the device and the user. The sound interaction allows the user, for example a farmer or a veterinarian, to obtain the status of various sensors and overall status of the livestock facility using voice commands and receiving voice based responses from the loudspeakers. The user's voice is captured by the microphones 701 which are then fed into a sound interaction module 702. The sound interaction module comprises a sound recognition module 702a and a sound synthesis module 702b. The user's voice captured by the microphones is fed to the sound recognition module 702a, and acts in this case as a voice recognition system. The module performs voice to text conversion to extract the words in the captured voice to determine the requirements of the user. The required information is fetched from the processing module which in turn provides the information by processing the various sensor output stored in the memory. The required information is later converted into sound by using a sound synthesis algorithm (in this case voice synthesis) and is played back to the user through the loudspeaker. Following are some of the example cases of where sound interaction is helpful.
A user, for example a farmer or a veterinarian, enters the livestock facility and asks about the health and/or welfare during the night or the change in health and/or welfare since his last visit. This can be done by speaking out a wake-up command to place the device in listening mode, followed by the ask-health and/or welfare-status command. The device answers with the information asked.
A user can ask for the status of a specific sensor (temperature, relative humidity, light, weather forecast . . . ) by using the wake-up command followed by the ask-sensor-status command. The answer will be played back by the loudspeaker

The sound interaction further enables two way interactions where the device can query the user and the responses can be stored in the device's rule based engine for future analysis. For example, the device can ask: ‘What is the heavy noise in the background’, the response received from the user is stored in the system for future analysis or for self-learning of the device.

In a similar way as the here-above described sound interaction mechanism between the device and the user, a sound interaction mechanism between the device and livestock animals is possible. A non-limiting example of a configuration to allow interaction of the device with livestock animals is the playing of calming sounds through the loudspeaker in response to detected aggression of the livestock animals.

FIG. 8

FIG. 8 illustrates a schematic representation of a system 800 for monitoring the status of a livestock facility 801, wherein the status of the livestock facility 801 includes health and/or welfare of livestock animals when inside the livestock facility 801 and/or management status of external systems 802-808 when installed in the facility 801. The system comprises a livestock facility 801 and a device 100 according to the first aspect of the present invention placed inside the livestock facility 801. For a description of the embodiment of the device shown in FIG. 8 is referred to the description of FIG. 1 above. As can be seen in FIG. 8, the device 100 is placed centrally inside the livestock facility 801 and is specifically attached to the ceiling 809 in a downwards-oriented fashion. Accordingly, the device 100 is ideally suited for monitoring the status of a livestock facility 801, wherein the status of the livestock facility 801 includes health and/or welfare of livestock animals inside the livestock facility and/or management status of external systems 802-808 installed in the facility 801. Inside the livestock facility 801, the following external systems are placed: feeding lines 802, water lines 803 as types of drinking systems, sprinklers 804, a ventilation system 805, a heating lamp 806 as a type of heating system, a pressure wash system 807 as type of cleaning system, and a radio 808 as a type of music system. For the monitoring by the device 100 of said status of the livestock facility 801 and for exemplary embodiments of the device intended for this purpose is referred to the discussion of any of the FIGS. 1-7 above. The system shown in FIG. 8 may also comprise a mobile apparatus, preferably a mobile phone, configured to receive output from the device, to store said output and to process said output. In the embodiment according to FIG. 8, the device 100 is placed centrally in an area with a diameter of 20 m (which is the area of the livestock facility 801 intended to accommodate livestock animals). For example, 200 to 250 pigs may be accommodated in an area with a diameter of 20 m. For example, 4000-6000 chickens may be accommodated in an area with a diameter of 20 m. Accordingly, the system 800 including the device 100 according to FIG. 8 is suitable for accommodating and monitoring 200 to 250 pigs or 4000-6000 chickens. In larger livestock facilities, multiple devices are required to monitor the livestock animals. For example, 4 devices in a typical pig farm with 1000 animals, and 5 devices in a typical chicken farm with 25000 animals.

The preceding description of the disclosed embodiments is provided to enable a person skilled in the art to make or use the present invention. Various modifications of these embodiments will be readily apparent to those skilled in the art, and the general principles defined herein may be applied to other embodiments without departing from the spirit or scope of the invention. Thus, the present invention is not intended to be limited to the embodiments shown herein but to be accorded the widest scope consistent with the following claims and the principles and features disclosed herein.

1. A device for monitoring the status of a livestock facility, wherein the status of the livestock facility includes health and/or welfare of livestock animals inside the livestock facility and/or management status of external systems installed in the facility, the device comprising:
i. a housing unit comprising:
a) one or more temperature sensors configured to measure the air temperature in the livestock facility;
b) one or more relative humidity sensors configured to monitor the relative humidity of the air in the livestock facility;
c) one or more light sensors configured to measure the color and/or the light intensity inside the livestock facility to distinguish the real or artificially imposed day and night regimes in the livestock facility;
d) one or more light emitting means configured to indicate the status of the device and/or abnormalities related to livestock animals inside the livestock facility;
e) one or more microphones; and
f) one or more loudspeakers; and
ii. a communication module comprising one or more wireless communication means to interact with other devices and/or external systems,
wherein two or more microphones are arranged in one or more circular peripheries and are configured to capture sounds produced in surrounding environment, wherein the two or more microphones enhance the accuracy of the device to perform additional functionalities, and wherein the loudspeaker of the housing unit is located at the centre of the circular periphery at equal distances from each of the microphones, such that the positioning of microphones and loudspeaker facilitates automated testing of microphones.

2. The device according to claim 1, wherein the device further comprises a memory configured to store output produced by the sensors and microphones enclosed in the housing unit.

3. The device according to claim 1, wherein the device further comprises a processing module for processing output received from the microphones to allow to identify the status of the livestock facility.

4. The device according to claim 1, wherein the device further comprises a sound interaction module configured to allow user interaction with the device, the interaction comprising capturing user's voice through a microphone and providing output related to the status of the livestock facility through the loudspeaker, and/or configured to allow interaction of the device with the livestock animals.

5. The device according to claim 4, wherein the sound interaction module is configured to allow microphone quality measurements by playing a predefined sound of known composition by the loudspeaker and recording the sound by the one or more microphones, resulting in one or more microphone signals, and determining the difference between the original sound played by the loudspeaker and the one or more microphone signals, or the correlation between the original sound and the one or more microphone signals, or, when multiple microphones are present, the correlation between pairs of microphone signals.

6. The device according to claim 1, wherein the one or more wireless communication means comprise a digital wireless data communication technology using sub-gigahertz radio frequency bands, wherein the digital wireless data communication technology using sub gigahertz radio frequency bands is LoRa.

7. The device according to claim 1, wherein the additional functionalities provided by the plurality of microphones comprise sound source localization, noise reduction, dereverberation, determining directionality of sound, and/or one or more advanced signal processing techniques selected from the group comprising neural networks and a beamforming operation.

8. The device according to claim 1, wherein the one or more light emitting means utilize different colors to indicate the status of the device and/or abnormalities related to the livestock animals, the different colors and meaning selected from the following:
a. a color to indicate that the device is not connected to the internet;
b. another color to indicate that the device is online and the status is ok;
c. yet another color to indicate a potential disease outbreak in the livestock facility;
d. even yet another color to indicate an intermediate state indicating the need for increased vigilance.

9. The device according to claim 1, wherein the livestock facility further comprises a gateway configured as the internet access point, enabling the creation of a wireless communication means mesh network automatically detecting and connecting all devices within reach of the gateway, either directly or indirectly through other devices, or the creation of a network wherein the devices are physically connected to the gateway by a plurality of Ethernet cables, wherein the gateway is equipped with one or more data storage means to store raw audio recording.

10. The device according to claim 1, wherein loudspeaker may be utilized to play any type of sound/music for the livestock animals and/or workers, wherein the sound/music is selected from the list comprising a calming sound for the livestock animals, classical music, natural vocalizations of the mother animal and music to improve the work environment of the workers.

11. The device according to claim 1, wherein the device further comprises a microprocessor and wherein the loudspeaker in combination with one or more microphones are configured to measure the acoustics of the livestock facility by means of said microprocessor, which microprocessor is configured to run an algorithm which can determine acoustics from sounds, and which microprocessor on the basis of the acoustics is configured to distinguish a big livestock facility from a smaller livestock facility.

12. The device according to claim 1, wherein the sound interaction module further allows two way interactions between the user and the device, where the device can ask questions to the user and store a received answer for future reference, and/or where the user can ask questions to the device and receive an answer from the device.

13. The device according to claim 1, wherein the housing unit further includes one or more antennas to communicate with other devices, gateway or external systems, wherein the one or more antennas are placed in a perpendicular plane compared to the position of the one or more microphones to minimize interference noise on the one or more microphones.

14. A system for monitoring the state of a livestock facility, wherein the status of the livestock facility includes health and/or welfare of livestock animals when inside the livestock facility and/or
management status of external systems when installed in the facility, comprising:
a livestock facility; and
one or more devices according to any one of the preceding claims 1 to 13 suitable to be placed inside the livestock facility,
wherein two or more of said devices are distributed inside the livestock facility.

15. The system according to claim 14, wherein one device for monitoring the status of a livestock facility, wherein the status of the livestock facility includes health and/or welfare of livestock animals inside the livestock facility and/or management status of external systems installed in the facility, the device comprising:
i. a housing unit comprising:
a) one or more temperature sensors configured to measure the air temperature in the livestock facility;
b) one or more relative humidity sensors configured to monitor the relative humidity of the air in the livestock facility;
c) one or more light sensors configured to measure the color and/or the light intensity inside the livestock facility to distinguish the real or artificially imposed day and night regimes in the livestock facility;
d) one or more light emitting means configured to indicate the status of the device and/or abnormalities related to livestock animals inside the livestock facility;
e) one or more microphones; and
f) one or more loudspeakers; and
ii. a communication module comprising one or more wireless communication means to interact with other devices and/or external systems,
wherein two or more microphones are arranged in one or more circular peripheries and are configured to capture sounds produced in surrounding environment, wherein the two or more microphones enhance the accuracy of the device to perform additional functionalities, and wherein the loudspeaker of the housing unit is located at the centre of the circular periphery at equal distances from each of the microphones, such that the positioning of microphones and loudspeaker facilitates automated testing of microphones
wherein the device is placed inside the livestock facility at level of each area inside the livestock facility with a diameter of between 16 and 24 m which is intended to accommodate livestock animals.
